(19) United States
(12) Patent Application Publication
(10) Pub. No.: US 2021/0362156 A1
(43) Pub. Date: Nov. 25, 2021

(54) SEPARATIONS OF FOCUSED PARTICLE FLOWS

(71) Applicant: HEWLETT-PACKARD DEVELOPMENT COMPANY, L.P., Spring, TX (US)

(72) Inventors: Viktor Shkolnikov, Palo Alto, CA (US); Chien-Hua Chen, Corvallis, OR (US)

(21) Appl. No.: 16/606,241

(22) PCT Filed: Jul. 16, 2018
(86) PCT No.: PCT/US18/42266

§ 371 Date: Oct. 18, 2019

(51) Int. Cl. B01L 3/00

(52) U.S. Cl. CPC B01L 3/502776; B01L 3/502761; B01L 2300/0816; B01L 2300/0645; B01L 2200/0652

(57) ABSTRACT

An example of an apparatus includes a focusing region to focus a sheath fluid and a particle flow. The apparatus also includes a particle inlet to inject the particle flow into the focusing region. In addition, the apparatus includes a sheath inlet to inject the sheath fluid into the focusing region. Also, the apparatus includes a cut-out disposed on a wall of the sheath inlet to distribute the sheath fluid about the particle flow to focus the particle flow into a linear stream. The apparatus further includes a separation region to apply a force to the particle flow. The force is dependent on a characteristic of a particle in the particle flow.

### BACKGROUND

Separating particles may be used in various industries, such as biology and medicine. For example, rare particles may be extracted from common particles using various separation techniques. In some examples, separation techniques may involve applying a force to the particles that are dependent on a property or characteristic of each particle such that particles with different properties or characteristics are moved varying amounts.

Manipulation of particles, such as biological particles which may include DNA, virus, bacteria, cell, multicellular organisms, etc., is used in various biomedical and biotechnological applications. There are designs of microfluidic and nanofluidic platforms to manipulate biological particles, which vary in size, such as from about 10 nm to about 100 μm. For example, some designs use electrokinetic forces. Microfluidic and nanofluidic platforms may provide a relatively small physical size, low power consumption, short reaction time, low cost, versatility in design, portability, reproducibility, and parallel operation and integration with other miniaturized devices. In addition, electrokinetic based manipulation may also provide label-free manipulation.

### BRIEF DESCRIPTION OF THE DRAWINGS

Reference will now be made, by way of example only, to the accompanying drawings in which:

FIG. 1 is a schematic diagram of an example apparatus to separate particles;

FIG. 2 is cross section view of the wall from the example shown in FIG. 1 along the line 2-2;

FIG. 3 is a schematic diagram of another example apparatus to separate particles with electric fields;

FIG. 4 is a schematic diagram of another example apparatus to separate particles;

FIGS. 5a-c are examples of (a) a centered square particle inlet in a focusing region, (b) a concentric particle inlet in a focusing region, and (c) a cross section view of the openings of a sheath inlet with a plurality of openings; and

FIG. 6 is flowchart of an example method to separate particles.

### DETAILED DESCRIPTION

In some examples, microfluidic and nanofluidic platforms use dielectrophoretic force to manipulate colloids, inert particles, and biological microparticles, such as red blood cells, white blood cells, platelets, cancer cells, bacteria, yeast, microorganisms, proteins, DNA, etc. Dielectrophoretic force is a specific electrokinetic technique has been used for trapping, sorting, focusing, filtration, patterning, assembly, and separating biological particles and entities suspended in a buffer medium. Dielectrophoretic forces acting on particles depend on various parameters, for example, charge of the particle, geometry of the device, dielectric constant of the medium and particle, and physiology of the particle.

In such microfluidic and nanofluidic platforms, the particles are generally focused such that the particles form a stream. In a present example, the stream of particles may pass into an electric field that applies a dielectrophoretic force to each particle in the stream. Since the absolute amount of force applied to each particle is different, the deviation of each particle from the stream will be different as well. Accordingly, the particles may subsequently be separated based on the deviation from the original path of the stream. In order to measure the deviation from the original path of each particle, focusing the particles into a narrow stream from which the deviation may be measured will provide more accurate separations.

As used herein, any usage of terms that suggest an absolute orientation (e.g. “top”, “bottom”, “vertical”, “horizontal”, etc.) are for illustrative convenience and refer to the orientation shown in a particular figure. However, such terms are not to be construed in a limiting sense as it is contemplated that various components will, in practice, be utilized in orientations that are the same as, or different than those described or shown

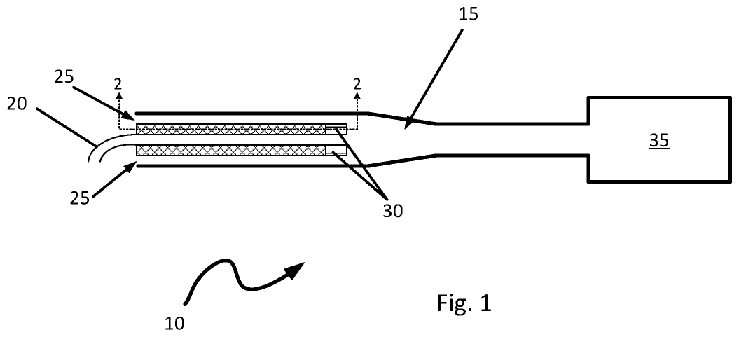

Referring to FIG. 1, an apparatus to separate particles with the application of a force, such as a dielectrophoretic force is shown at 10. The apparatus 10 is to receive a plurality of particles in a medium that via a particle flow. In addition, the apparatus 10 also receives a sheath fluid. In the present example, the apparatus 10 includes a focusing region 15, a particle inlet 20, a sheath inlet 25 having a wall with a cut-out 30, and a separation region 35.

The focusing region 15 is to focus a sheath fluid and a particle flow. The manner by which the focusing region 15 operates is not particularly limited and may be varied depending on the specific application, such as the specific fluids that flow through the apparatus 10. In the present example, the focusing region 15 is a tapered section to narrow the cross section of the channel in which the fluids flow. By narrowing the cross section, a particle flow is to be focused further along a line.

In the present example, the focusing region 15 includes an end of the channels from which the particle flow and the sheath fluid are combined. In particular, the focusing region 15 includes the cut-outs on the walls of the sheath inlet. For example, the focusing region 15 may direct the particle flow and the sheath fluid into channels that are about 750 μm deep by about 90 μm wide. It is to be appreciated that the depth and the width of the channels may be varied depending on the specific application. For example, the channels may have a depth of about 20 μm in some examples to 2 mm in other examples. The channels may have a width of about 5 μm in some examples and 100 μm in other examples. Furthermore, the taper is not limited and may be a wide range of angle since the flow is converging and the risk of flow separation is low.

The particle inlet 20 is to receive a particle flow and inject the particle flow into the focusing region 15. The particle flow includes a plurality of particles of interest. The particles of interest are not limited and may include red blood cells, white blood cells, platelets, cancer cells, bacteria, yeast, microorganisms, proteins, DNA and other biological microparticles. In particular, the particle flow may include two or more biological microparticles, such as red blood cells and platelets, for separation. As another example, white blood cells may be separated from tumor cells to provide an indication or diagnosis of cancer which may include downstream analysis of the cells with sequencing of nucleic acid contents. Further examples may include separation of bacterial cells from blood in sepsis, for downstream analysis of bacterial cells (or even detection of presence). Another example may be separation of fetal cells (including fetal nucleated red blood cells) in maternal blood, for downstream processing (e.g., sequencing) for determination of genetic disorders in the fetus (e.g. polyploidy)

The source of the particle flow is not particularly limited. For example, the particles of interest may be suspended in a fluid stored in a reservoir. The particle inlet 20 may then draw the fluid into the apparatus 10 with a pump (not shown) or other means. In other examples, the particles of interest may be received from an external dispensing mechanism or directly from a sample collected from a patient. The flow rate at which the particle flow is not particularly limited. In the present example, the particle flow rate is about 0.01 mL/min. In other examples, the rate may be increased to about 10 mL/min or decreased to about 0.001 μL/min. It is to be appreciated that other examples having different geometries may allow for rates outside of this range.

The sheath inlet 25 is to receive a sheath fluid and inject the sheath fluid into the focusing region 15. In the present example, the sheath fluid is a buffer compatible with separation, such as a low conductivity pH 7 buffer, that is made to be isotonic to the cells via sucrose. For example, the buffer may be a solution of about 9.5% sucrose, about 0.1 mg/mL dextrose, about 0.1% pluronic F68, about 0.1% bovine serum albumin, about 1 mM phosphate buffer pH 7 (adjustable), about 0.1 mM CaAcetate, about 0.5 mM MgAcetate, and about 100 units/ml catalase.

In the present example, the wall of the sheath inlet 25 includes a cut-out 30 disposed thereon at the end that extends into the focusing region 15. The cut-out 30 is to distribute the sheath fluid injected into the focusing region 15 about the particle flow. It is to be appreciated that as the sheath fluid surrounds the particle flow, the sheath fluid is to focus the particle flow into a linear stream. In particular, the cut-out 30 allows the sheath fluid to focus the particle flow in two dimensions since the sheath fluid to provide a linear stream of particle flow.

It is to be appreciated that the flow rate of the sheath fluid is not particularly limited and may be varied to control the focusing of the particle flow. In the present example, the sheath fluid flows at about 0.2 mL/min. In other examples, the rate may be increased to about 20 mL/min or decreased to about 0.001 mL/min. Accordingly, in this example, the sheath fluid flows at a rate approximately 20 times of the flow rate for the particle flow. In other examples, the relative flow rates may be adjusted. For example, the flow rate of the sheath fluid may be decreased to about 16 times the flow rate of the particle flow. In other examples, the flow rate of the sheath fluid may be set to a value to provide a ratio to particle flow from about 1:1 to about 100:1.

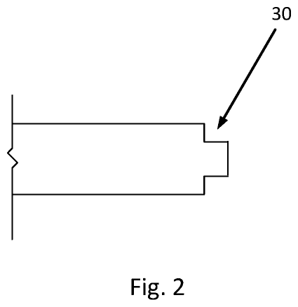

Referring to FIG. 2, a cross section of a wall of the sheath inlet 25 is illustrated. In this example, the cut-out 30 is shown to have a step profile. It is to be understood that the cut-out 30 is not particularly limited and may include other profiles. For example, the cut-out 30 may have a sloped profile, or a curved profile. The dimensions of the cut-out 30 are also not limited. In the present example, the cut-out 30 is about 100 μm deep (i.e. from the top and bottom of the channel). In other examples, the cut-out 30 may be about 10 μm deep to about 250 μm deep.

The length of the cut-out 30 may also be varied. For example, the cut-out 30 may be about 350 μm in length. In other examples, the cut-out 30 may be about 700 μm or about 1050 μm in length. The exact dimensions may be adjusted depending on various operating conditions that may affect the fluid flow in the system. In operation, the cut-out 30 in combination with the wall geometry in the focusing region 15 direct the sheath flow on top and bottom of the cell flow to sandwich the particle flow, and first focus the particles flow in the vertical direction with the walls of the sheath inlet 25. Then the cut-outs 30 allow sheath fluid to enter the particle flow channel at the top and bottom to sandwich the particle flow in the horizontal direction and to focus the particle flow in a horizontal direction resulting in a particle flow focused in both vertical and horizontal directions to provide a linear stream. The focusing of the particle stream into a linear stream provides for the particles entering the separation region 35 to have substantially similar velocities to decrease the variation of the hydrodynamic force between the particles to provide for better separations in the separation region 35.

The separation region 35 is to separate particles in the particle stream which may have difference characteristics or physical properties. In the present example, the separation region 35 applies varying forces to particles in the particle flow. The amount of force applied to a specific particle may be dependent on a specific characteristic or physical property. For example, the particles may be passed through an electric field where different particles respond differently to the electric field and thus subjected to a different force. In addition, different particles may have varying masses such that the amount of deviation from an original particle flow path may be varied. After the force is applied to each particle in the particle flow, the deviation from the original particle flow path may be measured. In some examples, the particles may be collected at a location to collect like particles that were subjected to the same deviation from the original particle flow path.

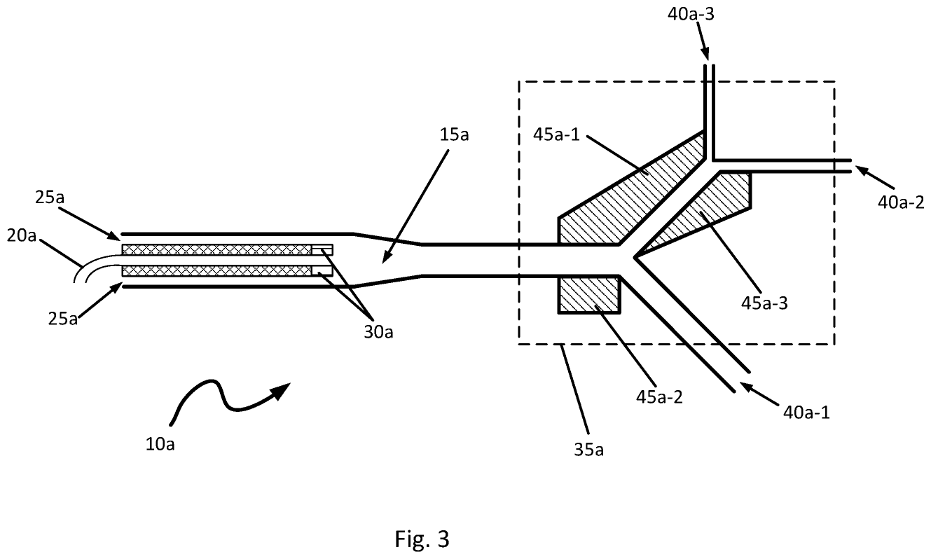

Referring to FIG. 3, another example of an apparatus to separate particles with the application of a force, such as a dielectrophoretic force is shown at 10a. Like components of the apparatus 10a bear like reference to their counterparts in the apparatus 10, except followed by the suffix “a”. The apparatus 10a includes a focusing region 15a, a particle inlet 20a, a sheath inlet 25a having a wall with a cut-out 30a, and a separation region 35a.

In the present example, the apparatus 10a also includes a plurality of outlets 40a-1, 40a-2, and 40a-3 (generically, these outlets are referred to herein as “outlet 40a ” and collectively they are referred to as “outlets 40a ”, this nomenclature is used elsewhere in this description). The outlets 40a are for removing particles from the separation region 35a once the particles have been separated. Accordingly, each of the outlets 40a are to remove a portion of the particle flow entering the separation region 35a from the focusing region 15a.

The separation region 35a is to separate particles in the particle stream which may have difference characteristics or physical properties. In the present example, the separation region 35a includes a plurality of electrodes 45a-1, 45a-2, and 45a-3 to apply varying forces to particles in the particle flow at different locations to direct portions of the particle flow to one of the outlets 40a. The amount of force applied to a specific particle may be dependent on a specific characteristic or physical property. Accordingly, the force applied to the particles will determine into which channel the portion of the particle flow may be directed.

In the present example, the electrode 45a-1 may be at a positive voltage compared to the electrode 45a-2, which may be a ground. Accordingly, the potential difference between the electrode 45a-1 and the electrode 45a-2 generates and electric field across the channel therebetween. In the present example, the geometry of the electrode 45a-1 and the electrode 45a-2 may generate a uniform electric field across the channel such that the linear stream of particles may travel through the electric field in a perpendicular direction. Accordingly, as the particles pass through the electric field between the electrode 45a-1 and the electrode 45a-2, different particles in the particle flow may respond differently to the electric field and thus subjected to a different force. Accordingly, a portion of the particle flow may be directed to the outlet 40a-1 as the particle flow passes through the electric field. The remaining portion of the particle flow may then be directed into the other channel. In other examples, the remaining portion may be directed to another outlet 40a to be collected after the separation region 35a.

Referring back to the present example shown in FIG. 3, the separation region 35a further includes another electrode 45a-3 proximate to the electrode 45a-1, such that the electrode 45a-1 and the electrode 45a-3 may interact to generate another electric field therebetween. In this example, the electrode 45a-3 may be at a negative voltage compared to the ground electrode 45a-2. In the present example, the geometry of the electrode 45a-1 and the electrode 45a-3 may generate a uniform electric field across the channel such that the portion of the particle flow having passed through a first electric field between the electrode 45a-1 and 45a-2 travel through a second electric field. Similar to the first electric field, the particles will pass through the second electric field in a perpendicular direction. Accordingly, as the particles pass through the electric field between the electrode 45a-1 and the electrode 45a-3, different particles in the portion of the original particle flow may respond differently to this second electric field and thus subjected to a different force. Accordingly, the portion of the particle flow may be split between the outlet 40a-1 and the outlet 40a-2. The remaining portion of the particle flow may then be directed into the other channel. In other examples, the remaining portion may be directed to another outlet 40a to be collected after the separation region 35a.

In operation, once the linear stream of particles enters the separation region 35a, the linear stream of particles passes through a first electric field between the electrodes 45a-1 and 45a-2. As the stream of particles passes through the first electric field, the particle flow is separated into two portions. A first portion is directed to the outlet 40a-1 where the portion of the particle flow exits the separation region 35a. In some examples, the particles passing through the outlet 40a-1 may be collected using container.

Continuing with this example, the second portion of the particle flow that is not directed to the outlet 40a-1 may be directed to a second electric field between the electrodes 45a-1 and 45a-2 for a second separation process. The second portion of the particle flow passes through the first electric field where the portion is further subdivided into two portions. A portion from the subdivision may be directed to the outlet 40a-2 where the portion of the particle flow exits the separation region 35a. The remainder of the second portion of the particle flow may then be directed to the outlet 40a-3 where the remainder exits the separation region 35a. In some examples, the particles passing through the outlets 40a-2 and 40a-3 may also be collected using container. For example, in the case of separating cells from a sample with cancer cells, the first step may involve separating tumor cells from the normal cells using the electric field between the electrodes 45a-1 and 45a-2 followed by a second step that may involve separating different types of tumor cells using the electric field between the electrodes 45a-1 and 45a-3.

It is to be appreciated that variations are contemplated. For example, although the present example illustrates two electric fields for separation, it is to be appreciated that the two portions of the channel where separation may occur is not limited to two electric fields. Other methods such as deterministic lateral displacement, acoustic cell separation, pinched flow fractionation, and Dean flow fractionations may be used in one of the areas. As another example of a variation, more or less than 2 separations may be included in the invention. For example, additional separations may be carried out on subsequent portions of the particle flow. As yet another example of a variation, although the present example may generate uniform magnetic fields between the electrodes 45a, other examples may not generate a uniform electric field.

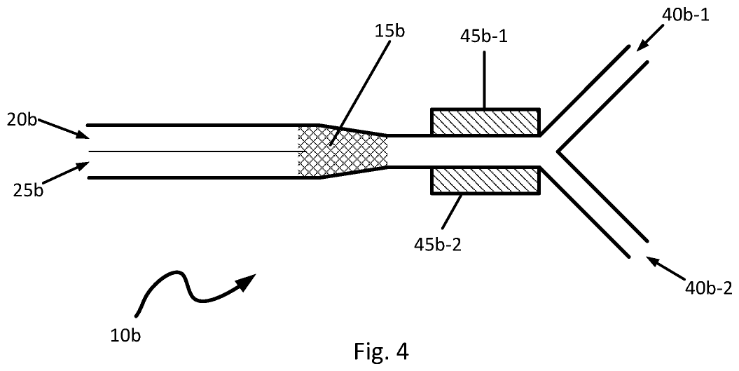

Referring to FIG. 4, another example of an apparatus to separate particles with the application of a force, such as a dielectrophoretic force is shown at 10b. Like components of the apparatus 10b bear like reference to their counterparts in the apparatus 10, except followed by the suffix “b”. The apparatus 10b includes a focusing region 15b, a particle inlet 20b, a sheath inlet 25b, a first electrode 45b-1, and a second electrode 45b-2.

The focusing region 15b is to focus a sheath fluid and a particle flow. The manner by which the focusing region 15b operates is not particularly limited and may be varied depending on the specific application, such as the specific fluids that flow through the apparatus 10b. In the present example, the focusing region 15b is a tapered section to narrow the cross section of the channel in which the fluids flow. By narrowing the cross section, a particle flow is to be focused further along a line.

In the present example, the particle inlet 20b is to receive a particle flow and inject the particle flow into the focusing region 15b. The focusing region 15b may include a shaped end of the particle inlet 20b. The shape of the end of the particle inlet 20b is not particularly limited. Since the particle flow is generally surrounded by the sheath fluid, the particle inlet 20b is generally disposed at approximately the center of a channel in the apparatus 10b. However, in some examples the particle inlet 20b may be offset from the center.

The sheath inlet 25b is to receive a sheath fluid and inject the sheath fluid into the focusing region 15b. The focusing region 15b may include a shaped end of the sheath inlet 25b. The shape of the end of the sheath inlet 25b is not particularly limited and is to distribute the sheath fluid about the particle flow to focus the particle flow into a linear stream. Accordingly, the sheath fluid is generally to surround the particle fluid around the particle inlet 20b in the channel in the apparatus 10b to provide for focusing in multiple axes, such as the vertical axis as well as the horizontal axis. In some examples, the sheath inlet 25b may surround the particle inlet 20b completely and in other examples, the sheath inlet 25b may be divided into a plurality of openings or injection points in the focusing region 15b such that the plurality of openings or injection points surrounds the particle inlet 20b.

In the present example, the first electrode 45b-1 and the second electrode 45b-2 are disposed proximate to the particle flow through a channel. In particular, the first electrode 45b-1 and the second electrode 45b-2 are disposed at opposite sides of the channel. The first electrode 45b-1 and may be at a positive voltage compared to the electrode 45b-2, which may be a ground or a negative voltage. Accordingly, the potential difference between the first electrode 45b-1 and the second electrode 45b-2 generates an electric field across the channel therebetween. In the present example, the geometry of the electrode 45b-1 and the electrode 45b-2 is to generate a uniform electric field across the channel such the linear stream of particle flow travels through the electric field in a perpendicular direction. Accordingly, as the particles in the particle flow pass through the electric field between the electrode 45b-1 and the electrode 45b-2, different particles in the particle flow may respond differently to the electric field and thus subjected to a different force dependent on a characteristic of the particle in the particle flow. Accordingly, a portion of the particle flow may be directed to the outlet 40b-1 as some particles in the particle flow passes through the electric field. The remaining particles of the particle flow may then be directed into the outlet 40b-2.

The apparatus 10b may include a plurality of outlets 40b-1 and 40b-2 The outlets 40b are for removing particles from the apparatus 10b once the particles have been separated from a particle flow. Accordingly, the outlets 40b-1 and 40b-2 are to remove separate portions of the particle flow entering the apparatus 10b via the particle inlet 20b after being separated by the electric field between the first electrode 45b-1 and the second electrode 45b-2.

It is to be appreciated that variations are contemplated. For example, although the present example illustrates two electrodes generating a single electric field for separation, it is to be appreciated that the separation method is not limited to use of electric fields. In addition, as another example of a variation, more than a single electric field may be used for separations such that three or more portions may be separated from the particle flow using additional electrodes to generate additional electric fields through which the particle flow is to pass.

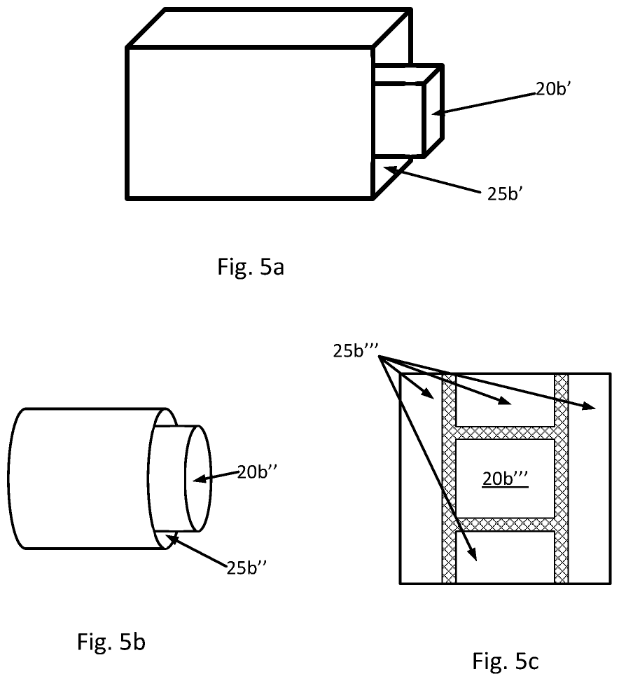

Referring to FIGS. 5a-c, various shapes of the ends of the particle inlet 20b and the sheath inlet 25b are illustrated. Referring to FIG. 5a, the particle inlet 20b′ is dispose in approximately in the center of the sheath inlet 25b′. In this example, both the particle inlet 20b′ and the sheath inlet 25b′ are generally square in shape. In other examples, the particle inlet 20b′ and the sheath inlet 25b′ may be rectangular in shape or may have another shape altogether. The manner by which the particle inlet 20b′ is centered within the sheath inlet 25b′ is not particularly limited. For example, the apparatus 10b may provide a crossover region where the smaller particle inlet 20b′ enters into the sheath inlet 25b′ via a sealed point. In other examples, the particle inlet 20b′ and the sheath inlet 25b′ may be combined prior to entering the apparatus 10b.

Furthermore, it is to be appreciated that the particle inlet 20b′ is not limited to being centered within the sheath inlet 25b′ and may be offset in other examples. In addition, the particle inlet 20b′ may be adjustable to vary the location of the linear stream of particle flow in the channel to steer the linear stream of particle flow or adjust the height within the channel.

Referring to FIG. 5b, another shape of the ends of a particle inlet 20b″ and a sheath inlet 25b″ is illustrated. It is to be appreciated that the example shown in FIG. 5b is similar to the example shown in FIG. 5a, except the shape of the particle inlet 20b″ and the sheath inlet 25b″ are circular and that the particle inlet 20b″ is concentric with the sheath inlet 25b″. It is to be appreciated that the particle inlet 20b″ is not limited to being concentric with the sheath inlet 25b″ and may be offset in other examples. Furthermore, the particle inlet 20b″ may be adjustable to vary the location of the linear stream of particle flow in the channel to steer the linear stream of particle flow or adjust the height within the channel.

Referring to FIG. 5c, a sheath inlet 25b′″ divided into a plurality of opening to surround a particle inlet 20b′″ is illustrated. In this example, the particle inlet 20b′″ does not enter the sheath inlet 25b′″ via a crossover region. Instead, the sheath inlet 25b′″ is divided to surround the particle inlet 20b′″. It is to be appreciated that the flow of sheath fluid from each opening of the sheath inlet 25b″ may not be equal. Furthermore, the flow of sheath fluid from each opening may be adjustable to vary the direction of the linear stream of particle flow in the channel to steer the linear stream of particle flow. The manner by which the flow from each opening of the sheath inlet 25b′″ is adjusted is not particularly limited. For example, the flows may be adjusted by adjusting the geometry of the openings or by using various active elements such as pumps for each subchannel leading to the opening.

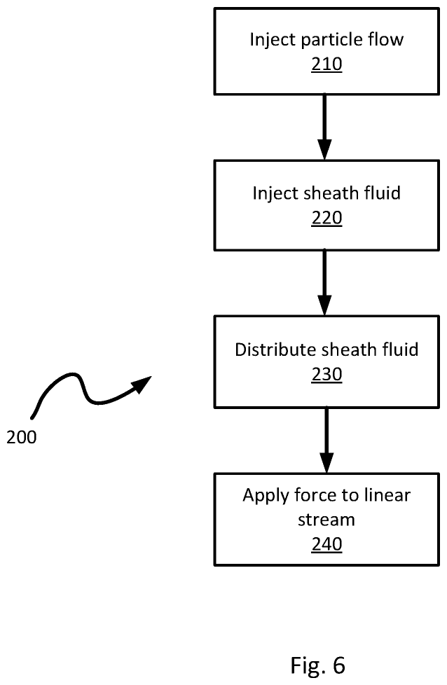

Referring to FIG. 6, a flowchart of a method to separate particles is shown at 200. In order to assist in the explanation of method 200, it will be assumed that method 200 may be performed with any of the apparatus 10, 10a, or 10b described above. Indeed, the method 200 may be one way in which apparatus 10, 10a, or 10b may be configured to separated particles. Furthermore, the following discussion of method 200 may lead to a further understanding of the apparatus 10, 10a, or 10b and their various components. For purposes of the following discussion, it is to be assumed that the method 200 is carried out on the apparatus 10. Furthermore, it is to be emphasized, that method 200 may not be performed in the exact sequence as shown, and various blocks may be performed in parallel rather than in sequence, or in a different sequence altogether.

Beginning at block 210, a particle flow is injected into the focusing region 15 via the particle inlet 20. In the present example, the particle flow includes a plurality of particles of interest in a mixture for separation. The particles of interest are not limited and may include red blood cells, white blood cells, platelets, cancer cells, bacteria, yeast, microorganisms, proteins, DNA and other biological microparticles. In particular, the particle flow may include two or more biological microparticles, such as red blood cells and platelets, for separation. As another example, white blood cells may be separated from tumor cells to provide an indication or diagnosis of cancer.

Block 220 involves a sheath fluid to be injected into the focusing region 15 via the sheath inlet 25. It is to be appreciated that in this example, the sheath fluid is generally injected at the same time as the particle flow in a continuous manner to provide a consistent flow and mixture of particle flow with sheath fluid. For example, the sheath inlet 25 may provide sheath fluid at a rate about sixteen times the rate at which particle flow is provided via the particle inlet.

Block 230 may distribute that sheath fluid around the particle flow using cut-outs 30 that are disposed on a wall of the sheath inlet 25. In particular, it is to be appreciated that the cut-outs 30 direct the flow of the sheath fluid for focus the particle flow into a linear stream. The manner by which the sheath fluid focuses the particle flow is not limited. For example, the cut-out 30 in combination with the wall geometry in the focusing region 15 direct the sheath flow on top and bottom of the particle flow to sandwich the particle flow, and first focus the particles flow in the vertical direction. Then sheath fluid sandwiches the particle flow in the horizontal direction and to focus the particle flow in a horizontal direction resulting in a particle flow focused in both vertical and horizontal directions to provide a linear stream.

It is to be appreciated that in other examples, other configurations of the focusing region may provide for alternative methods to focus the particle flow into a linear stream. For example, the focusing region 15b may be used where the particle inlet 20b and the sheath inlet 25b may have other configurations to focus the particle flow in more than one dimension into a linear stream.

Block 240 applies a force to the linear stream of particle flow. In particular, the force applied varies and is dependent on a characteristic or physical property of each particle in the linear stream of particles. Accordingly, the application of a force may provide for the ability to separate or sort particles based on the characteristic, such as the response of a particle to an electric field. Furthermore, it is to be appreciated that once a portion of the particle flow is separated, the portion may be directed to an outlet for removal from the apparatus 10, such as for sample collection purposes.

It should be recognized that features and aspects of the various examples provided above may be combined into further examples that also fall within the scope of the present disclosure.

What is claimed is:

1. An apparatus comprising:
a focusing region to focus a sheath fluid and a particle flow;
a particle inlet to inject the particle flow into the focusing region;
a sheath inlet to inject the sheath fluid into the focusing region;
a cut-out disposed on a wall of the sheath inlet to distribute the sheath fluid about the particle flow to focus the particle flow into a linear stream; and
a separation region to apply a force to the particle flow, wherein the force is dependent on a characteristic of a particle in the particle flow.

2. The apparatus of claim 1, further comprising a first outlet to remove a first portion of the particle flow after the separation region.

3. The apparatus of claim 2, wherein the separation region includes a first electrode and a second electrode to generate a first electric field therebetween, wherein the first portion is separated and directed to the first outlet as the particle flow passes through the first electric field.

4. The apparatus of claim 3, further comprising a second outlet to remove a second portion of the particle flow after the separation region, wherein the second portion is separated from the first portion by the force.

5. The apparatus of claim 4, wherein the separation region includes a third electrode proximate to the first electrode to generate a second electric field therebetween, wherein the second portion is separated and directed to the second outlet as the particle flow passes through the second electric field.

6. The apparatus of claim 5, wherein the first electric field and the second electric field are uniform.

7. An apparatus comprising:
a particle inlet to inject a particle flow;
a sheath inlet to inject a sheath fluid to focus the particle flow, wherein the sheath fluid is distributed about the particle flow to focus the particle flow into a linear stream;
a first electrode disposed proximate to the particle flow; and
a second electrode to be paired with the first electrode to generate a first electric field therebetween to apply a force to the particle flow, wherein the force is dependent on a characteristic of a particle in the particle flow to separate the particle, wherein the first electric field is uniform.

8. The apparatus of claim 7, further comprising a first outlet to remove a first portion of the particle flow.

9. The apparatus of claim 8, wherein the first portion is separated and directed to the first outlet by the first electric field.

10. The apparatus of claim 9, further comprising a third electrode proximate to the first electrode to generate a second electric field therebetween, wherein a second portion of the particle flow is separated as the particle flow passes through the second electric field.

11. The apparatus of claim 7, wherein the sheath inlet comprises a plurality of openings to surround the particle inlet.

12. The apparatus of claim 7, wherein the particle inlet is concentric with the sheath inlet.

13. A method comprising:
injecting a particle flow into a focusing region via a particle inlet;
injecting a sheath fluid into the focusing region via a sheath inlet;
distributing the sheath fluid around the particle flow with a cut-out disposed on a wall of the sheath inlet, wherein the cut-out directs flow of the sheath fluid, wherein the sheath fluid is to focus the particle flow into a linear stream; and
applying a force to the linear stream, wherein the force is dependent on a characteristic of a particle in the linear stream.

14. The method of claim 13, wherein applying the force separates a portion of a plurality of particles in the linear stream.

15. The method of claim 14, further comprising directing the portion to an outlet for removal.
